(19) United States
(12) Patent Application Publication
(10) Pub. No.: US 2004/0213859 A1
(43) Pub. Date: Oct. 28, 2004

(54) Organic nutrient for hair loss treatment

(76) Inventor: Brian D. Zelickson, Minneapolis, MN (US)

Correspondence Address: John F. Dolan, Fredrikson & Byron, P.A., 4000 Pillsbury Center, 200 South Sixth Street, Minneapolis, MN, 55402-1425, US

(21) Appl. No.: 10/207,566

(22) Filed: Jul. 29, 2002

Related U.S. Application Data
(63) Continuation of application No. 09/711,172, filed on Nov. 9, 2000, now abandoned.
Application No. 09/711,172 is a continuation of application No. 09/637,097, filed on Aug. 10, 2000, now abandoned.
(60) Provisional application No. 60/154,570, filed on Sep. 17, 1999.

(51) Int. Cl. A61K 35/78

(52) U.S. Cl. 424/725

(57) ABSTRACT

The present invention relates to a composition which is generally useful for the treatment of skin and hair. More specifically, the composition is useful for the treatment of androgenetica, also known as androgenetic alopecia or pattern hair loss. In general, the composition of the present invention comprises one or more phytoestrogens, one or more natural herbal extracts, and optionally a pharmaceutically and/or dermatologically acceptable carrier. Other embodiments of the present invention comprise one or more phytoestrogens, one or more natural herbal extracts, one or more substances selected from the group consisting of vitamins, preferably b-complex vitamins, minerals and brewer's yeast and optionally a pharmaceutically and/or dermatologically acceptable carrier.

### CROSS-REFERENCES TO RELATED APPLICATIONS

This application claims priority to and incorporates by reference the entire contents of both, U.S. Provisional Application No. 60/154,570 filed Sep. 17, 1999 and U.S. application Ser. No. 09/637,097 filed Aug. 10, 2000.

### FIELD OF THE INVENTION

This invention relates to a composition which is generally useful for the treatment of skin and hair. More specifically, the composition is useful for the treatment of androgenetica, also known as androgenetic alopecia or pattern hair loss. In general, the composition of the present invention comprises one or more phytoestrogens, one or more natural herbal extracts, and optionally a pharmaceutically and/or dermatologically acceptable carrier. Other embodiments of the present invention comprise one or more phytoestrogens, one or more natural herbal extracts, one or more substances selected from the group consisting of vitamins, preferably b-complex vitamins, minerals and brewer's yeast and optionally a pharmaceutically and/or dermatologically acceptable carrier.

### BACKGROUND OF THE INVENTION

Alopecia has been a problem afflicting mankind and animals for thousands of years. In many individuals, alopecia causes embarrassment, psychological problems, including depression, and can affect one's self image and feelings of sexuality. Alopecia has been hypothesized to have various etiologies in males and females, many of which involve gonadal steroids. The loss of hair from the scalp is more common in men (e.g., male pattern baldness or androgenic alopecia) than in women (e.g., female pattern baldness). Cosmetic research has devoted millions of dollars and countless hours of research to solve this problem.

Human hair undergoes a normal growth cycle commonly known as the pilar cycle. During the pilar cycle, hair forms, grows and falls out, before being replaced by a new hair shaft, which appears in the same follicle. Humans typically have about 100,000 to 150,000 hairs on their scalps, and it is normal to lose about 50 to 150 hairs daily.

The pilar cycle can be broken down into three successive phases: the anagen phase the catagen phase and the telogen phase. During the anagen phase, the hair undergoes a period of active growth associated with an intensive metabolic activity in the bulb. The subsequent catagen phase is transitory and marked by a slowing-down of mitotic activity. The final telogen phase corresponds to a period of rest for the follicle, with the hair being shed. The hairs on the head are always in different stages of the cycle, so it is normal to loose scalp hair everyday.

It also must be explained that hair growth depends on whether the hair growth selected for treatment is androgen-stimulated hair growth (e.g., beard hair and torso hair generally in humans) or hair growth that is not androgen-stimulated (e.g., scalp hair in humans). Administration of an antiandrogen formulation topically in a dermatologically acceptable vehicle to an area of skin having androgen-stimulated hair growth or by administering the formulation by oral means, injection, suppository or other sublingual forms in general causes a reduction in hair growth. Topical application of the compound in a dermatologically acceptable vehicle to an area of skin having hair growth (i.e., from the scalp) that is reduced in the presence of androgens or by administering the formulation by oral means, injection, suppository or other sublingual forms, (e.g., because of androgenic alopecia) in general causes an increase in hair.

In male pattern hair loss, the normal hair growth cycle is disrupted and more than the average number of hairs are shed per day without having the old hairs replaced by new ones. Male pattern hair loss is determined by a combination of male hormones (androgens) and heredity. Men susceptible to male pattern baldness usually experience the onset sometime in their 20's and it becomes more common as they age. Androgenetic alopecia is the most common type of hair loss in men, with approximately 50% of men experiencing this hair loss to some degree by the age of 50.

In addition to adrogenetic alopecia, other factors may influence hair loss, many of which are temporary. Amongst these factors include stress of an illness or major surgery, medicines, such as those used in chemotherapy, blood thinners, antidepressants, excessive amounts of vitamin A and certain disease states like diabetes.

A number of pharmaceutical treatments have been proposed for treating male pattern hair loss, such as Minoxidil. Minoxidil is applied topically to the scalp and has been shown to stimulate hair growth in individuals with androgenetic alopecia. Another medication utilized for treatment of adrogenetic alopecia is Finisteride. Finisteride, a prescription antiandrogen medication, is an inhibitor of type II 5-alpha-reductase and has been shown to be effective in decreasing scalp DHT (dihydrotestosterone) by inhibiting conversion of testosterone to DHT. Scientists believe that DHT contributes to the shortening of the growth phase and thinning of the hair. Oral administration of Finisteride slowed hair loss, increased hair growth and improved the appearance of hair. Other anitandrogen medications which interfere with DHT binding at hormone receptor sites on hair follicle cells are Spironolactone, Cyproterone acetate, Estrogens and Cimetidine.

Pharmaceutical treatments have various disadvantages which deter their use. Limited results in the treatment of hair loss is one such disadvantage. Also, side effects, such as diminished sexual function, has been attributed to various pharmaceutical treatments. Finally, the high cost of long-term treatment with pharmaceutical treatments has deterred their widespread use.

Another medicant believed to treat hair loss and stimulate hair growth is the use of herbal extracts or a combination of multiple herbal extracts. See for example, U.S. Pat. No. 5,972,345. Many herbal extracts, which have been found to be useful in the treatment of benign prostatic hyperplasia, are further believed to have a natural affect on the skin and hair. One such herbal extract is the extract of the berries of Saw Palmetto. Saw Palmetto berries contain an oil with a variety of fatty acids and phytosterols. The fat soluble extract of Saw Palmetto berries has been shown to inhibit the conversion of testosterone, which is thought to be responsible for the enlargement of the prostrate. In addition, Saw Palmetto extract inhibits the binding of DHT to receptors, thus blocking DHT's action and promoting the breakdown of the potent compound. Other herbal extracts, such as African Pygeum and Stinging Nettles Extract have been known as having potential in stimulating hair growth and more generally improving condition of the hair and skin.

The disadvantage of the utilization of herbal extracts or a combination of such extracts are that they fail to provide all of the necessary components to provide the optinmum treatment for hair loss. Furthermore, herbal extract formulations often do not provide an adequate proportion of each individual component which provides an efficient coaction among the various components.

### SUMMARY OF THE INVENTION

In general, the composition of the present invention comprises one or more phytoestrogens, one or more natural herbal extracts, and optionally a pharmaceutically and/or dermatologically acceptable carrier. Other embodiments of the present invention comprise one or more phytoestrogens, one or more natural herbal extracts, one or more substances selected from the group consisting of vitamins, preferably b-complex vitamins, minerals and brewer's yeast and optionally a pharmaceutically and/or dermatologically acceptable carrier.

It been discovered that embodiments of the composition of the present invention are generally useful for all types of skin and hair treatment. As previously mentioned, various embodiments of the composition are useful for the treatment of hair loss, and is especially useful for treatment of androgenetic alopecia. Furthermore, embodiments of the composition also may be utilized for the reduction or elimination of hair in areas of the human body which are androgen stimulated (i.e. the face or torso). Also, embodiments of the composition are effective in the prevention and treatment of acne.

One embodiment of the present invention comprises 80-99.5% herbal extract and 0.5-20% Phytoestrogens. Another preferred embodiment of the present invention comprises 85-95% herbal extract, 0.01-3% Biotin, 0.1-5% Thiamin, 0.1-5% Pyridoxine, 0.05-3% Folic Acid, 0.1-5% Riboflavin, 0.5-7% Niacinamide, and 0.5-8% Phytoestrogens. The percentage amounts reflect the “by mass” percentages and do not consider the amount of carrier, if a carrier is added to the composition.

The composition can be administered by various means including topical application, oral administration, injection, sublingual administration or any other systematic form of administration.

These and other aspects of the invention will be evident upon reference to the following detailed description.

### BRIEF DESCRIPTION OF THE DRAWINGS

FIG. 1 depicts the top view of the head of subject #1 following administration of the present invention for a period of one month.

FIG. 2 depicts the top view of the head of subject #1 following administration of the present invention for a period of three months.

FIG. 3 depicts the rear view of the head of subject #1 following administration of the present invention for a period of one month.

FIG. 4 depicts the rear view of the head of subject #1 following administration of the present invention for a period of three months.

FIG. 5 depicts a magnified view of a portion of the head of subject #1 following administration of the present invention for a period of one month.

FIG. 6 depicts a magnified view of the head of subject #1 following administration of the present invention for a period of three months.

FIG. 7 depicts the right side view of the head of subject #1 following administration of the present invention for a period of one month.

FIG. 8 depicts the right side view of the head of subject #1 following administration of the present invention for a period of three months.

FIG. 9 depicts the left side view of the head of subject #1 following administration of the present invention for a period of one month.

FIG. 10 depicts the left side view of the head of subject #1 following administration of the present invention for a period of three months.

FIG. 11 depicts the top view of the head of subject #2 following administration of the present invention for a period of one month.

FIG. 12 depicts the top view of the head of subject #2 following administration of the present invention for a period of three months.

FIG. 13 depicts the rear view of the head of subject #2 following administration of the present invention for a period of one month.

FIG. 14 depicts the rear view of the head of subject #2 following administration of the present invention for a period of three months.

TABLE 1 depicts the terminal hair counts of subjects 1 and 2.

TABLE 2 depicts the percentage increase in terminal hair counts at three months for subjects 1 and 2.

### DESCRIPTION OF THE INVENTION

As previously mentioned, the composition of the present invention comprises one or more phytoestrogens, one or more natural herbal extracts, and optionally a pharmaceutically and/or dermatologically acceptable carrier. Other embodiments of the present invention comprise one or more phytoestrogens, one or more natural herbal extracts, one or more substances selected from the group consisting of vitamins, preferably b-complex vitamins, minerals and brewer's yeast and optionally a pharmaceutically and/or dermatologically acceptable carrier. Each component of the composition is prepared according to the traditional procedures known in the art, then combined in a suitable composition for administration to the patient for treatment.

The combining of the various components may be performed by the simple mixture of all the components in a single pot operation. For example, adding one or more phytoestrogens to a mixing container, subsequently adding one or more herbal extracts to the same container and then mixing the individual components together. This process can be utilized in the mixture of all the various components described herein when preparing a composition of the present invention. It is noted that various embodiments of the present invention may be prepared utilizing other procedures which adequately provide a uniform distribution of the individual components throughout the composition.

The phytoestrogens referred to in the previously described composition of the present invention include, but are not limited to the following: 1) Isoflavones, such as genistein, daidzein, biochanin A, and formononetin; 2) Lignans, or Resorcyclic Acid Lactones, such as matairesinol, secoisolariciresinol, enterolactone and enterodiol; 3) coumestans, such as coumestrol and 4) Equol, which is an estrogenic substance that results when intestinal bacteria break down either formononetin or daidzein. It has been found that the composition's inclusion of phytoestrogens enhances, accelerates and controls the systematic involvement of all the substances, in particular the herbal extracts, included in the composition.

In one embodiment of the present invention, red clover is utilized as a source of phytoestrogens. Red clover can be used either by direct incorporation into the present composition or utilized to synthesize the phytoestrogens, which are subsequently incorporated into the composition. The incorporation of red clover directly into the composition also provides vitamin A, vitamin C, B-complex, calcium, chromium, iron and magnesium to the composition.

The composition also includes one or more anti-androgen herbal extracts. An herbal extract generally is the solution or preparation containing the active principles of the plant, herb, drug, juice or the like. Saw Palmetto, African Pygeum and Stinging Nettles Extract are examples of such herbal extracts. However, any herbal extract having anti-androgen characteristics may be used in the present invention.

One embodiment of the present invention includes Saw Palmetto as a component of the composition. As previously described Saw Palmetto is a small palm tree with large leaves and largedeep red black berries. Saw Palmetto berries contain an oil with a variety of fatty acids and phytosterols. The fat soluble extract of Saw Palmetto berries has been shown to inhibit the conversion of testosterone, which is thought to be responsible for the enlargement of the prostrate. In addition, Saw Palmetto extract inhibits the binding of DHT to receptors, thus blocking DHT's action and promoting the breakdown of the potent compound.

Another component utilized in various embodiments of the composition of the present invention are vitamins and preferably b-complex vitamins. Generally, these embodiments incorporate one or more of the following b-complex vitamins: Biotin, Thiamin, Pyridoxine, Folic Acid, Riboflavin, Niacinamide and Nicotinic acid. It is noted that present invention may also include the addition of other vitamins and minerals into the present composition.

Embodiments of the composition of the present invention may also include the addition of minerals and/or brewer's yeast. Brewers yeast is an excellent source of several nutrients including thiamin (vitamin B1), riboflavin (vitamin B2), nicotinic acid (Vitamin B3), pyridoxine (vitamin B6), pantothenic acid (Vitamin B5), biotin and folic acid, as well as some minerals and trace minerals, especially Chromium and Selenium. It also contains about 810 percent nucleic acid, which may have an immune enhancing effect.

As previously mentioned one embodiment of the present invention comprises 80-99.5% herbal extract and 0.5-20% Phytoestrogens as the active component of the composition. Additional embodiments of the present invention comprise as an active component of the composition 50-95% herbal extract, 0-10% Biotin, 0.1-15% Thiamin, 0.1-15% Pyridoxine, 0-10% Folic Acid, 0.1-15% Riboflavin, 0.5-20% Niacinamide, and 0.5-20% Phytoestrogens. The percentage amounts reflect the “by mass” percentages and do not consider the amount of carrier, if a carrier is added to the composition. A preferable embodiment of the active component of the composition comprises 85-95% herbal extract, 0.01-3% Biotin, 0.1-5% Thiamin, 0.1-5% Pyridoxine, 0.05-3% Folic Acid, 0.1-5% Riboflavin, 0.5-7% Niacinamide, and 0.5-8% Phytoestrogens. Again, the percentage amounts reflect the “by mass” percentages and do not consider the amount of carrier, if a carrier is added to the composition.

It has been discovered that embodiments of the composition are generally useful for all types of skin and hair treatment. As previously mentioned, the composition of the present invention is useful for the treatment of hair loss, and is especially useful for treatment of androgenetic alopecia. Furthermore, embodiments of the composition also may be utilized for the reduction or elimination of hair in areas of the human body which are androgen stimulated (e.g. the face or torso). Also as previously mentioned, embodiments of the composition of the present invention are also useful in treatment of acne. These embodiments of the composition of the present invention are effective in the treatment and prevention of the previously mentioned conditions due to their inhibiting characteristic in reducing androgen from reaching androgen receptor sites. The composition's active role in inhibiting androgen from reaching androgen receptor cites is linked to the treatment and prevention of hair loss from the scalp, the reduction of hair growth in androgen stimulated cites and the treatment and prevention of acne.

As previously mentioned, embodiments of the composition are suitable for providing an anti-androgenic effect when administered to a patient. The administration of such embodiments of the composition may be facilitated by topical application, oral administration, injection, suppository, sublingual forms or any other mode which is acceptable in administration of such compositions.

In one embodiment of the present invention, the composition is administered orally to a patient. Oral dosage unit compositions include tablets, capsules, liquids and other conventional oral forms. In a tablet the composition may be alone or present in an amount of from about 10-100 percent by weight, with the inert carrier constituting the remainder of the tablet. Typical pharmaceutically acceptable carriers include ingredients such as talcum, maize starch, polyvinyl pyrrolidone and lactose, together with a small amount of a tabletting agent such as magnesium stearate. As also mentioned, liquid oral dosage unit formulations may also be used in which the compositions are incorporated into vehicles conventionally used for lipid soluble compounds. Also, suppositories with the composition are also contemplated, to provide a rectal suppository administration of the drug, which form takes advantage of the usual suppository ingredients.

The high potency of the composition permits relatively low dosages both systemically, via oral or suppository routes, or through topical (transdermal) application. As previously mentioned the composition may be administered without the utilization of a pharmaceutically acceptable carrier. However, if a carrier is present, the concentration of the active composition is from about 10-100 percent by weight of the composition, and generally from about 40 to 50 percent, is useful. Topical application on an infrequent basis, through a sustained release delivery, may indicate a relatively higher amount of the compositions, preferably in the range of from about 50-100 percent by weight. A relatively lower concentration of the composition is indicated where a larger surface area is treated, such as the back, chest, etc., e.g., a concentration of from about 0.1-49 percent by weight.

Generally, a single oral dosage unit of the composition is administered as one oral dosage unit several times per day, in general up to about four times per day and preferably twice a day. For a normal adult male this comprises an amount of about 200 mg to 1000 mg per oral dosage unit and preferably from about 400 mg to 600 mg per oral dosage unit form. As previously mentioned the topical application of the composition may include a larger amount per topical dosage unit of the composition than the oral dosage unit. The general localization of the composition allows for a larger dosage to be administered.

The topical administration of the composition to a patient may also generally include a pharmaceutically and dermatologically acceptable carrier suitable for topical administration. Pharmaceutically acceptable carriers include, but are not limited to, an alcohol, salve, suspension, emulsion, ointment, cream, powder or spray. In a preferred embodiment, an alcohol such as ethanol and isopropanol are employed as pharmaceutically and dermatologically acceptable carriers. For deeper penetration the composition may be incorporated in liposomes or mixed with a penetration enhancer such as DMSO.

In another embodiment, the composition is provided in a sustained release composition for transdermal application to the skin of a patient. The sustained release carrier should be one which will maintain the composition at the skin and permit release to the skin for a period of preferably a minimum of about six to eight hours. An example of a sustained release carrier is polyvinyl alcohol with a molecular weight of at least 8,000. For example, a polyvinyl alcohol having a molecular weight of about 20,000 is suitable for use with the composition. However any suitable sustained release carrier may be utilized with the composition.

A further embodiment provides a skin composition suitable for topical administration to a patient to be exposed to ultraviolet light which includes both the composition of the present invention and an ultraviolet screening agent such as para-aminobenzoic acid or cocoa butter.

Another embodiment of the invention is the incorporation of the composition into a shampoo which is advantageously provided for sufferers of skin problems and particularly, male pattern baldness. The shampoo comprises conventional shampoo ingredients having incorporated therein the composition of the present invention.

The following examples depict testing performed utilizing various embodiments of the present invention.

### EXAMPLE 1

After informed consent was given a healthy Caucasian male subject (age 32) with male patterned alopecia volunteered for observation regarding hair growth promoting activity of the composition of the present invention. The composition of the invention utilized in the observation was as follows:

| | | |
|---|---|---|
| Saw Palmetto | 250 | mg |
| B-Complex Vitamins: | | |
| Biotin | 50 | μg |
| Thiamin Pyridoxine B6 | 3.0 | mg |
| Folic Acid | 200 | μg |
| Riboflavin | 2.6 | mg |
| Nicacinamide | 7 | mg |
| Phytoestrogens: | | |
| Daidzein | 2 | mg |
| Genistein | 85 | μg |
| Biochanin | 262 | μg |
| Formononetin | 209 | μg |

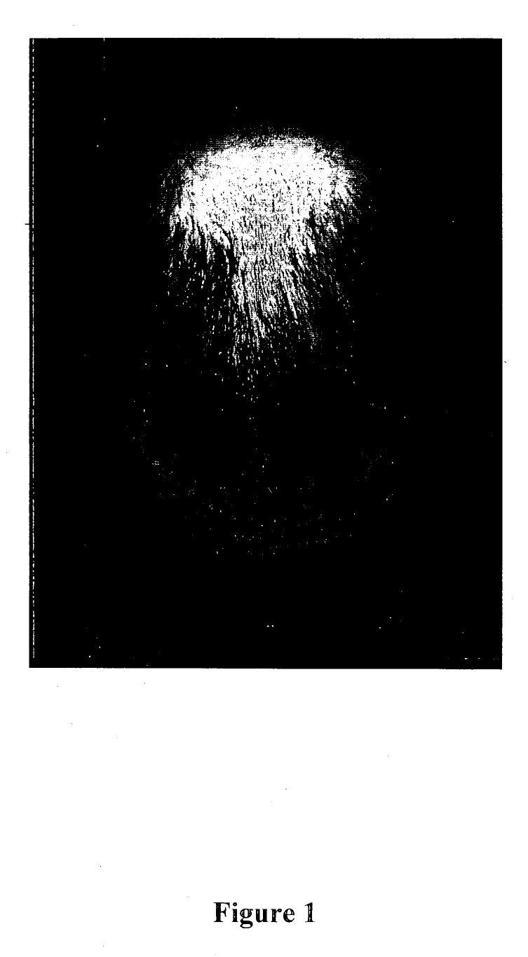

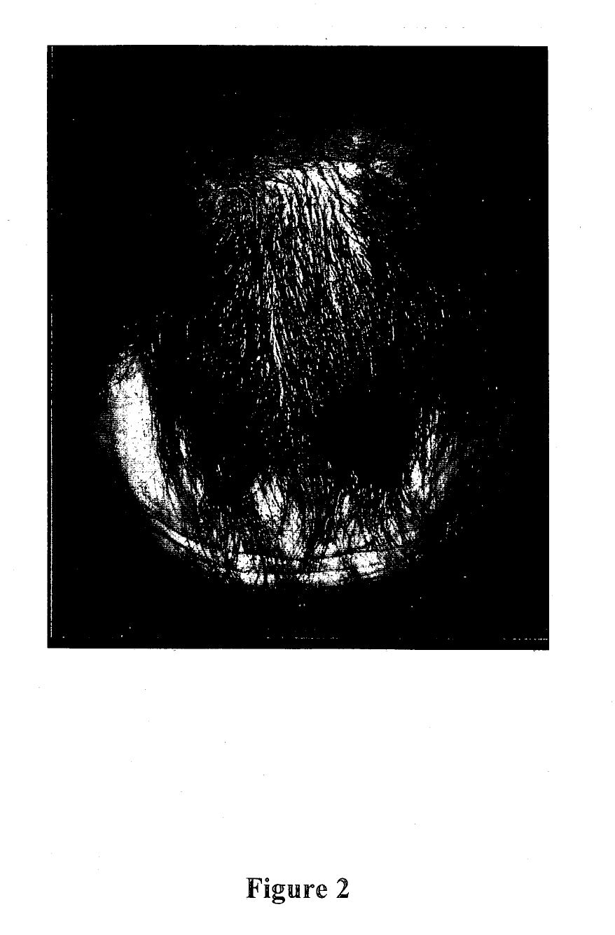

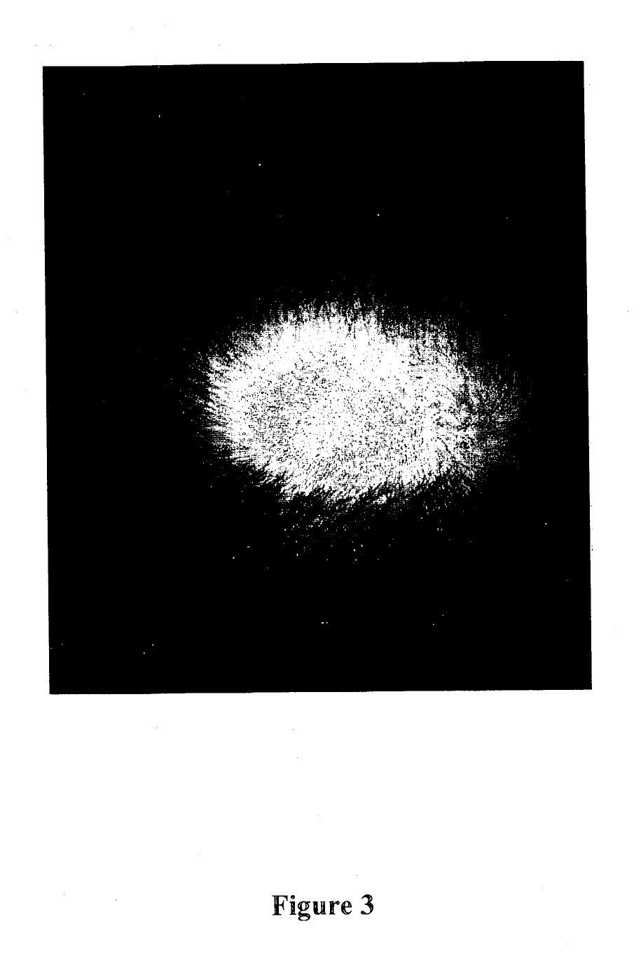

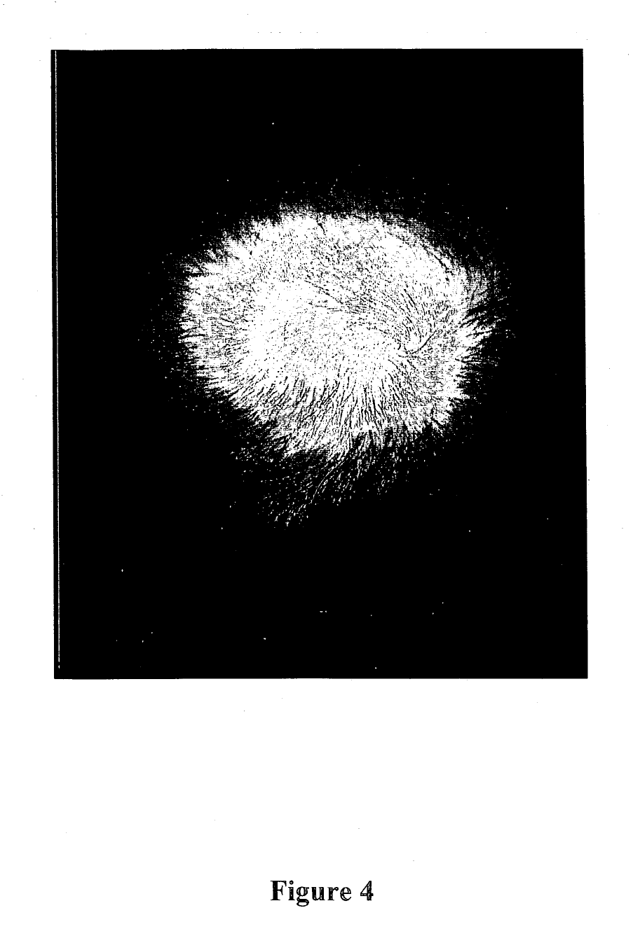

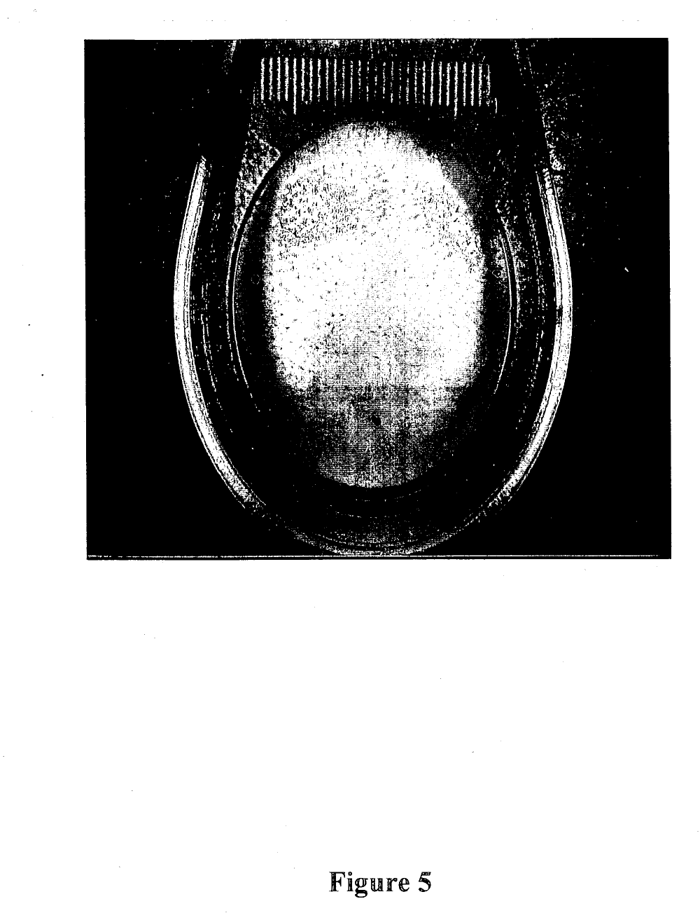

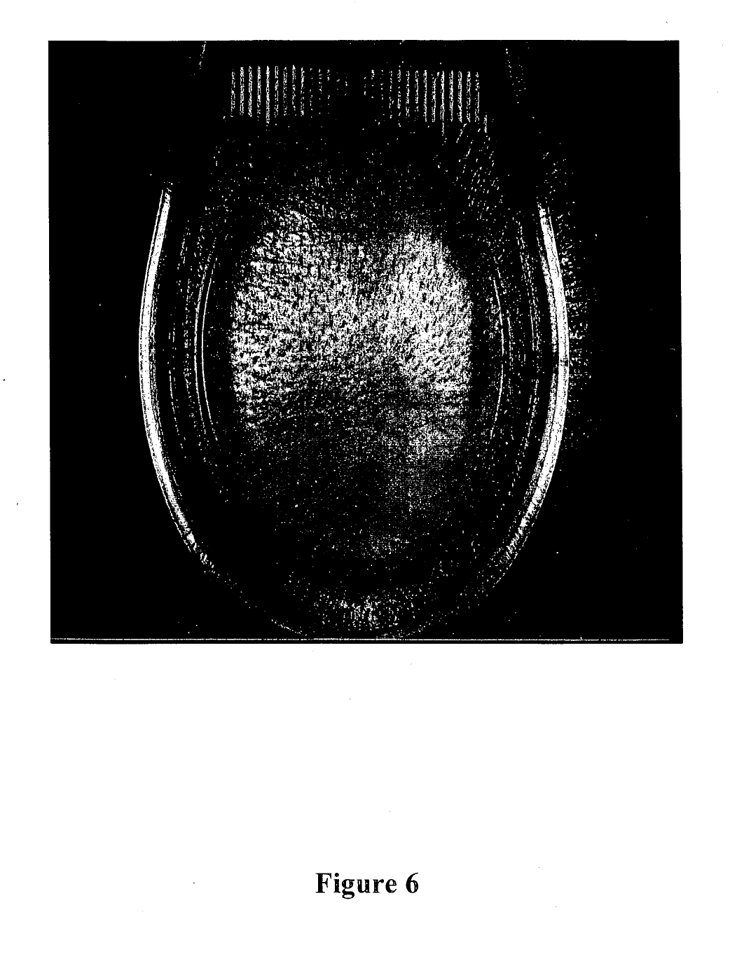

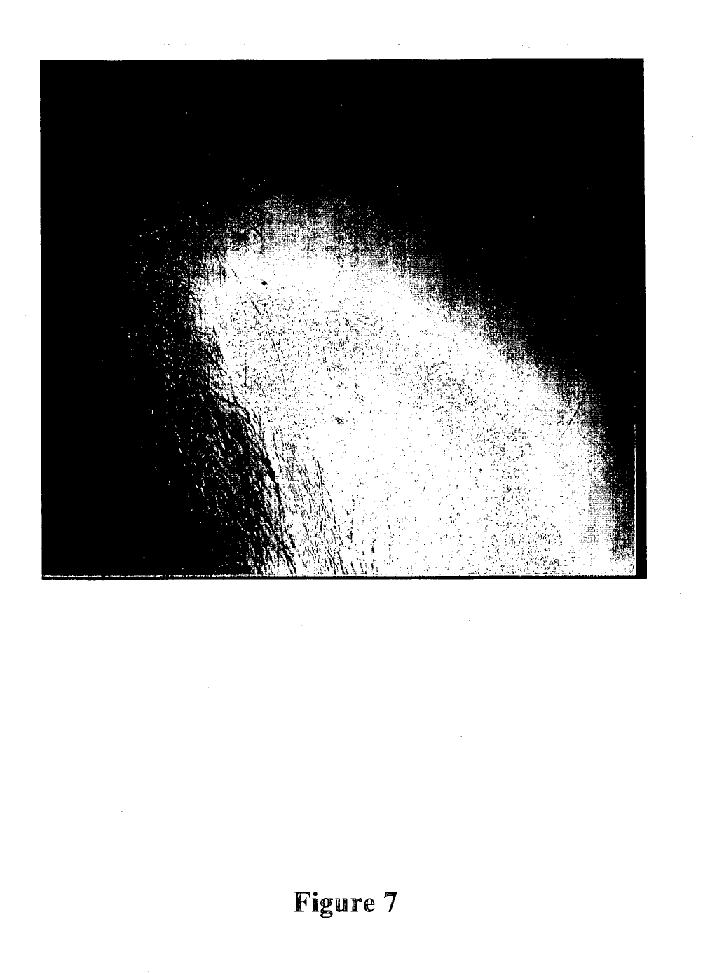

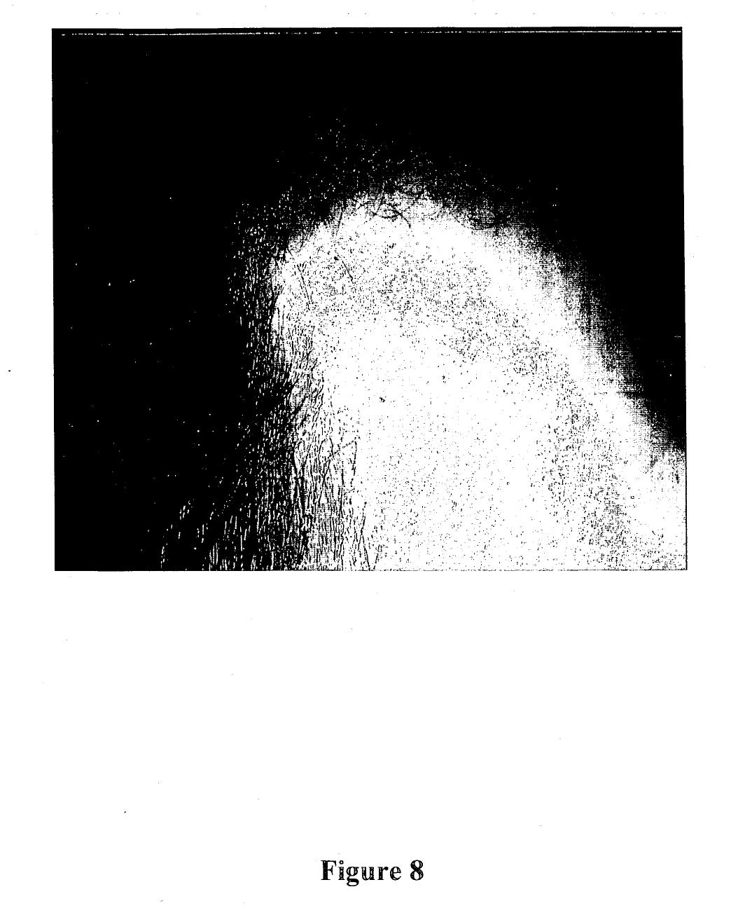

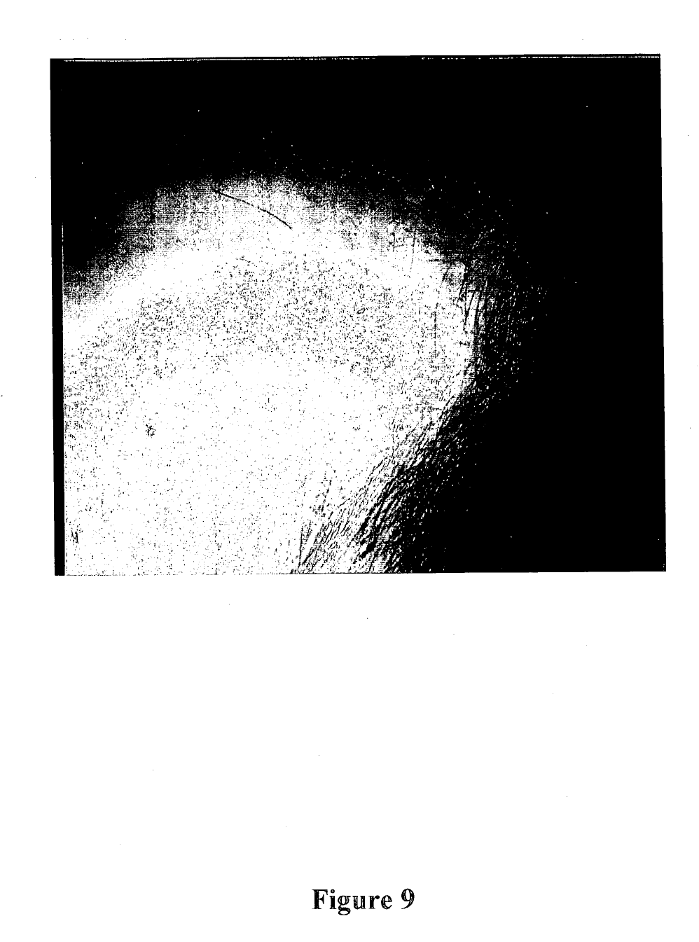

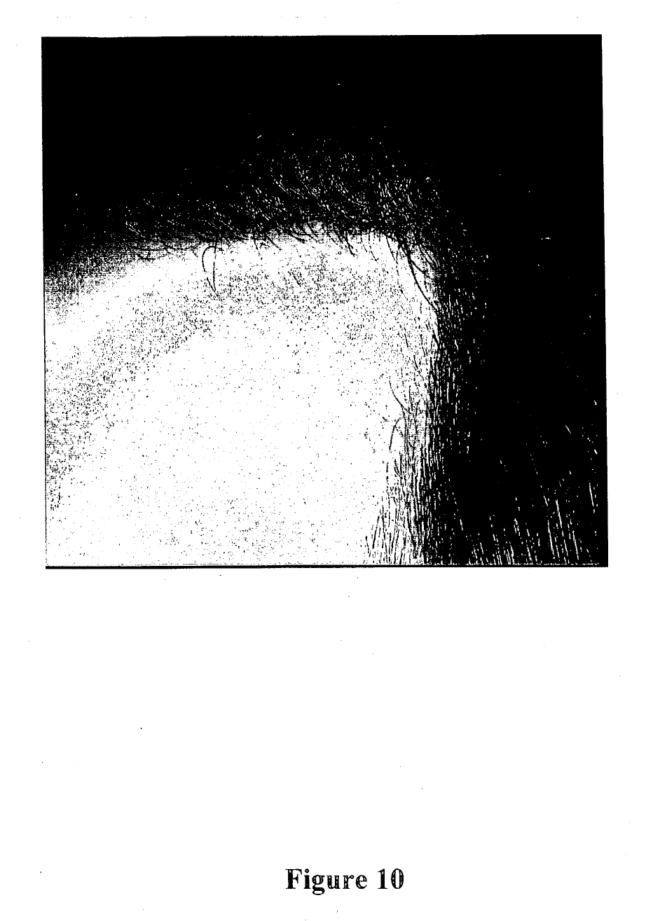

It was recorded that before taking the composition the patient had dramatic hair loss on the frontoparietal area of the head. The patient was instructed to take one unit dosage orally twice a day for a six month period. He was also instructed to report any potential side effects and any subjective changes in hair loss. At one month the patient noted a marked decrease in the amount of hair loss and also noted new hair growth. FIGS. 1, 3, 5, 7, and 9 depict the top, rear, left side, right side and magnified views of the patient's head at one month. The patient reported again at three months the marked decrease in hair loss and a further significant increase in the amount of new hair growth. FIGS. 2, 4, 6, 8 and 10 depict the top, rear, left side, right side and magnified views of the patient's head at three months which correspond to the respective areas shown in FIGS. 1, 3, 5, 7, and 9. It is noted that the patient reported a significant improvement in his acne since taking the composition. Furthermore, the patient did not experience any untoward side effects from taking the composition.

### EXAMPLE 2

After informed consent was given a healthy caucasian male subject (age 47) with male patterned alopecia volunteered for observation regarding hair growth promoting activity of the composition of the present invention. The composition of the invention utilized in the observation was as follows:

| | | |
|---|---|---|
| Saw Palmetto | 250 | mg |
| B-Complex Vitamins: | | |
| Biotin | 50 | μg |
| Thiamian Pyridoxine B6 | 3.0 | mg |
| Folic Acid | 200 | μg |
| Riboflavin | 2.6 | mg |
| Nicacinamide | 7 | mg |
| Phytoestrogens: | | |
| Daidzein | 2 | mg |
| Genistein | 85 | μg |
| Biochanin | 262 | μg |
| Formononetin | 209 | μg |

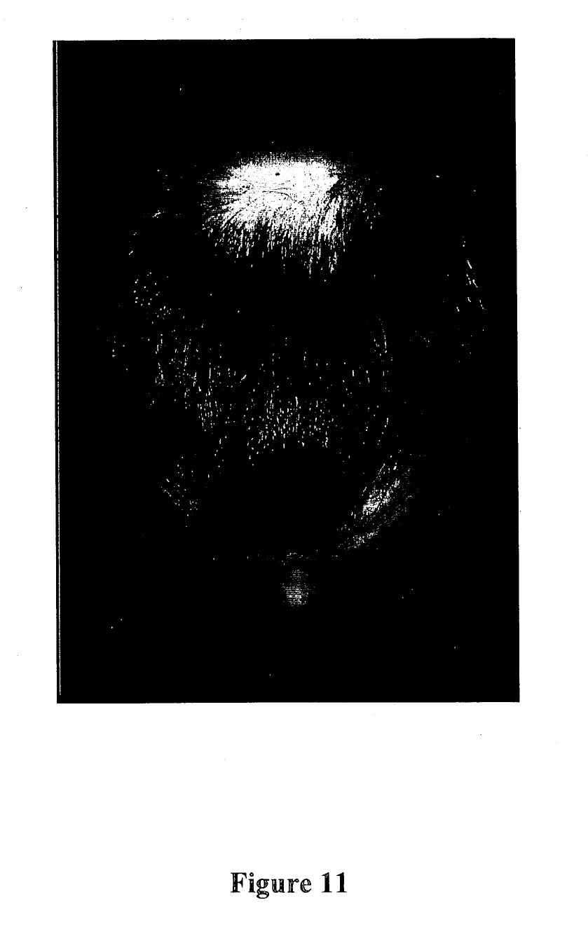

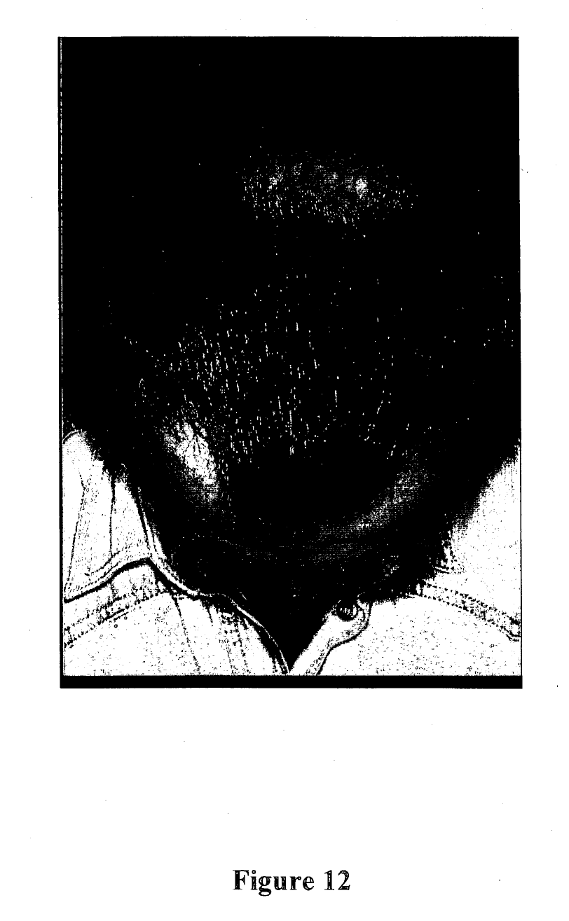

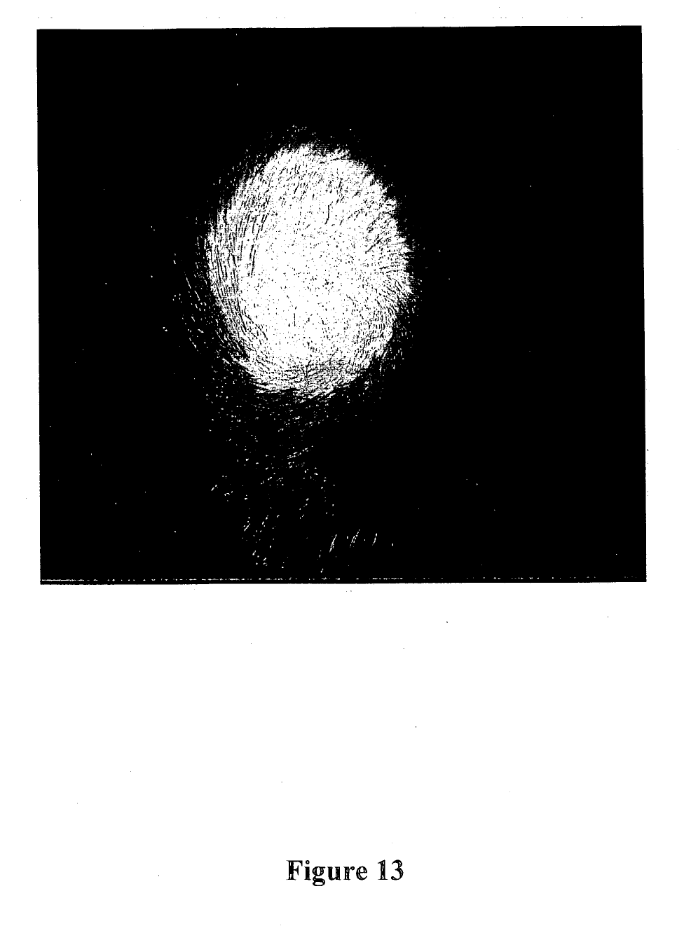

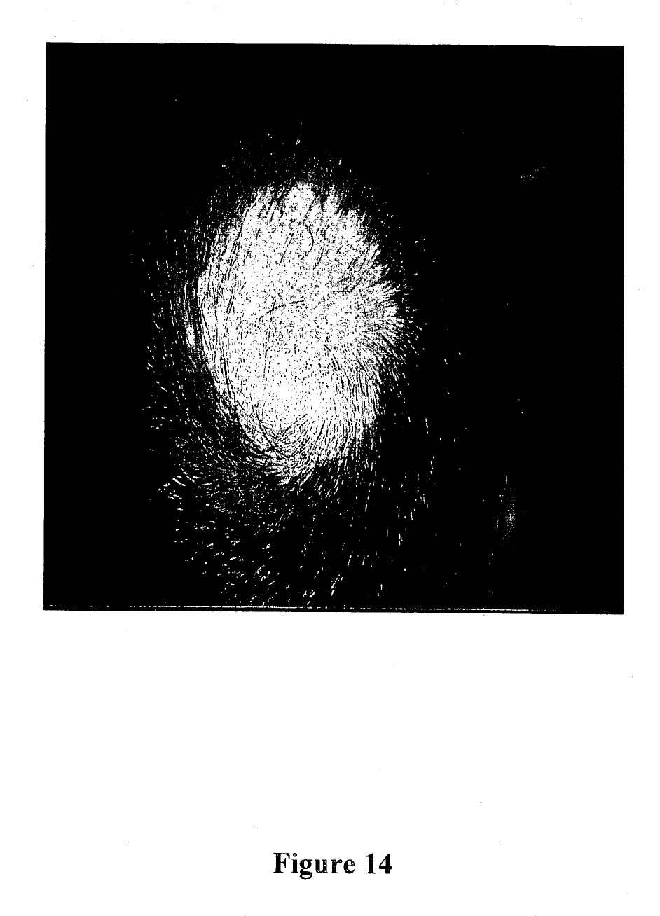

It was recorded that before taking the composition the patient had dramatic hair loss on the frontoparietal area of the head. The patient was instructed to take one unit dosage orally twice a day for a six month period. He was also instructed to report any potential side effects and any subjective changes in hair loss. At one month the patient noted a marked decrease in the amount of hair loss and also noted new hair growth. FIGS. 11 and 13 depict the top and rear views of the patient's head at one month. The patient reported again at three months the marked decrease in hair loss and a further significant increase in the amount of new hair growth. FIGS. 12, and 14 depict the top and rear view of the patient's head at three months, which correspond to the respective areas shown in FIGS. 11 and 13. The patient did not experience any untoward side effects from taking the composition. The patient did not experience any untoward side effects from taking the composition.

Table 1 depicts the hair count results for subjects 1 and 2. The table illustrates an increase of approximately 350 terminal hairs for subject #1 and 200 terminal hairs for subject #2 following three months of administration of the formulation described in the previously mentioned examples. The increase in terminal hair counts equates to approximately a 60% increase for subject #1 and a 175% increase for subject #2. These percentages are illustrated in Table 2.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations which fall within the spirit and broad scope of the invention.

What is claimed is:

1. A composition comprising one or more phytoestrogens, one or more herbal extracts and optionally a pharmaceutically acceptable carrier.

2. The composition of claim 1 further comprising one or more substances selected from the group consisting of vitamins, minerals and brewer's yeast.

3. The composition of claim 1 wherein the phytoestrogens are selected from a group consisting of genistein, daidzein, biochanin A, and formononetin.

4. The composition of claim 1 wherein the phytoestrogens are provided by red clover.

5. The composition of claim 1 wherein the herbal extracts are selected from a group consisting of saw palmetto, African pygeum and stinging nettles extract.

6. The composition of claim 2 wherein the vitamins are b-complex vitamins.

7. The composition of claim 6 wherein the b-complex vitamins are selected from a group consisting of biotin, thiamin, pyridoxine, folic acid, riboflavin, niacinamide and nicotinic acid.

8. The composition of claim 2 wherein the composition comprises one or more phytoestrogens, saw palmetto, one or more b-complex vitamins and optionally a pharmaceutically acceptable carrier.

9. The composition of claim 8 wherein an active component of the composition comprises 50-95% Saw Palmetto, 0-10% Biotin, 0.1-15% Thiamin, 0.1-15% Pyridoxine, 0-10% Folic Acid, 0.1-15% Riboflavin, 1-20% Niacinamide, and 0.5-20% Phytoestrogens.

10. The composition of claim 9 wherein the active component comprises 85-95% Saw Palmetto, 0.01-3% Biotin, 0.1-5% Thiamin, 0.1-5% Pyridoxine, 0.05-3% Folic Acid, 0.1-5% Riboflavin, 0.5-7% Niacinamide, and 0.5-8% Phytoestrogens.

11. The composition of claim 10 wherein the active component comprises 250 mg Saw Palmetto, 50 μg Biotin, 2.3 mg Thiamin, 3.0 mg Pyridoxine B6, 200 μg Folic Acid, 2.6 mg Riboflavin, 7 mg Nicacinamide, Daidzein 2 mg, Genistein 85 mcg, Biochanin 262 mcg, Formononetin 209 mcg.

12. The composition of claim 1 wherein an active component of the composition comprises 80-99.5% herbal extract and 0.5-20% Phytoestrogens.

13. The composition of claim 1 wherein the pharmaceutical carrier is DMSO.

14. The composition of claim 1 wherein the composition is incorporated in liposomes.

15. A method for the prevention and treatment of hair loss on the scalp of a mammal comprising administering to the mammal a therapeutically effective amount of a composition comprising one or more phytoestrogens, one or more herbal extracts and optionally a pharmaceutically acceptable carrier.

16. The method of claim 15 wherein the composition further comprises one or more substances selected from the group consisting of vitamins, minerals and brewer's yeast.

17. The method of claim 16 wherein an active component of the composition comprises 50-95% Saw Palmetto, 0-10% Biotin, 0.1-15% Thiamin, 0.1-15% Pyridoxine, 0-10% Folic Acid, 0.1-15% Riboflavin, 1-20% Niacinamide, and 1-20% Phytoestrogens.

18. The method of claim 17 wherein the active component comprises 85-95% Saw Palmetto, 0.01-3% Biotin, 0.1-5% Thiamin, 0.1-5% Pyridoxine, 0.05-3% Folic Acid, 0.1-5% Riboflavin, 0.5-7% Niacinamide, and 0.5-8% Phytoestrogens.

19. The method of claim 18 wherein the active component comprises 250 mg Saw Palmetto, 50 μg Biotin, 2.3 mg Thiamin, 3.0 mg Pyridoxine B6, 200 μg Folic Acid, 2.6 mg Riboflavin, 7 mg Nicacinamide, Daidzein 2 mg, Genistein 85 μg, Biochanin 262 μg and Formononetin 209 μg.

20. The method of claim 15 wherein an active component of the composition comprises 80-99.5% herbal extract and 0.5-20% Phytoestrogens.

21. The method of claim 15 wherein the composition is administered orally.

22. The method of claim 15 wherein the formulation is administered topically.

23. A method for the prevention, reduction and elimination of hair in androgen stimulated areas comprising administering to a mammal a therapeutically effective amount of a composition comprising one or more phytoestrogens, one or more herbal extracts and optionally a pharmaceutically acceptable carrier.

24. The method of claim 23 wherein the composition further comprises one or more substances selected from the group consisting of vitamins, minerals and brewer's yeast.

25. The method of claim 19 wherein an active component of the composition comprises 50-95% Saw Palmetto, 0-10% Biotin, 0.1-15% Thiamin, 0.1-15% Pyridoxine, 0-10% Folic Acid, 0.1-15% Riboflavin, 1-20% Niacinamide, and 0.5-20% Phytoestrogens.

24. The method of claim 25 wherein the active component comprises 85-95% Saw Palmetto, 0.01-3% Biotin, 0.1-5% Thiamin, 0.1-5% Pyridoxine, 0.05-3% Folic Acid, 0.1-5% Riboflavin, 0.5-7% Niacinamide, and 0.5-8% Phytoestrogens.

25. The method of claim 24 wherein the active component comprises 250 mg Saw Palmetto, 50 μg Biotin, 2.3 mg Thiamin, 3.0 mg Pyridoxine B6, 200 μg Folic Acid, 2.6 mg Riboflavin, 7 mg Nicacinamide, Daidzein 2 mg, Genistein 85 μg, Biochanin 262 μg and Formononetin 209 μg.

26. The method of claim 23 wherein an active component of the composition comprises 80-99.5% herbal extract and 0.5-20% Phytoestrogens.

27. The method of claim 23 wherein the composition is administered orally.

28. The method of claim 23 wherein the formulation is administered topically.

29. A method for the prevention, reduction and elimination of acne comprising administering to a mammal a therapeutically effective amount of a composition comprising one or more phytoestrogens, one or more herbal extracts and optionally a pharmaceutically and dermatologically acceptable carrier.

30. The method of claim 29 wherein the composition further comprises one or more substances selected from the group consisting of vitamins, minerals and brewer's yeast.

31. The method of claim 30 wherein an active component of the composition comprises 50-95% Saw Palmetto, 0-10% Biotin, 0.1-15% Thiamin, 0.1-15% Pyridoxine, 0-10% Folic Acid, 0.1-15% Riboflavin, 1-20% Niacinamide, and 0.5-20% Phytoestrogens.

32. The method of claim 31 wherein the active component comprises 85-95% Saw Palmetto, 0.01-3% Biotin, 0.1-5% Thiamin, 0.1-5% Pyridoxine, 0.05-3% Folic Acid, 0.1-5% Riboflavin, 0.5-7% Niacinamide, and 0.5-8% Phytoestrogens.

33. The method of claim 32 wherein the active component comprises 250 mg Saw Palmetto, 50 μg Biotin, 2.3 mg Thiamin, 3.0 mg Pyridoxine B6, 200 μg Folic Acid, 2.6 mg Riboflavin, 7 mg Nicacinamide, Daidzein 2 mg, Genistein 85 μg, Biochanin 262 μg and Formononetin 209 μg.

34. The method of claim 29 wherein an active component of the composition comprises 80-99.5% herbal extract and 0.5-20% Phytoestrogens.

35. The method of claim 29 wherein the composition is administered orally.

36. The method of claim 29 wherein the formulation is administered topically.
